(19) United States
(12) Patent Application Publication
(10) Pub. No.: US 2024/0081738 A1
(43) Pub. Date: Mar. 14, 2024

(54) Nail Sensing Based, Wireless Multi-Modal Tonoarteriogram Monitoring Apparatus And System

(71) Applicant: Hong Kong Centre for Centre for Cerebro-Cardiovasculer Health Engineering Limited, Hong Kong (CN)

(72) Inventors: Yuanting ZHANG, Hong Kong (CN); Ting XIANG, Hong Kong (CN); Nan Ji, Hong Kong (CN); Yunhung Gao, Hong Kong (CN)

(21) Appl. No.: 18/070,490

(22) Filed: Nov. 29, 2022

(30) Foreign Application Priority Data
Sep. 8, 2022 (CN) 202211098163.9

(51) Int. Cl. A61B 5/00; A61B 5/021

(52) U.S. Cl. CPC A61B 5/6826; A61B 5/002; A61B 5/02108; A61B 5/6833; A61B 5/6843; A61B 5/721; A61B 5/7267; A61B 5/742; A61B 5/02427

(57) ABSTRACT

The present invention relates to a wireless multi-modal tonoarteriogram (TAG) monitoring apparatus and system based on nail sensing. The invention relates to the field of blood pressure monitoring technology. The nail sensing based, wireless multi-modal tonoarteriogram monitoring apparatus comprises a nail patch, a plurality of sensors and a processing module, at least one first sensor of the plurality of sensors is arranged on the nail patch, the plurality of sensors are used for obtaining a biological signal at a fingernail; the processing module is used for obtaining a target physiological parameter according to the biological signal at fingernail, with the target physiological parameter comprising a central tonoarteriogram signal. Compared with the prior art by placing a finger cuff on a finger to obtain a biological signal, the invention uses the nail patch to adhere onto a nail which is light weight, good in appearance, and is convenient to wear by a user without affecting normal activities of the user which realizes a long-term monitoring of the user. At the same time, when compared with the placing of a finger cuff on a finger to obtain a biological signal, the use of a nail patch will not be affected by the pulling of skin to thereby obtain biological signal of higher quality.

### FIELD OF THE INVENTION

The present invention relates to the field of blood pressure monitoring technology, specifically, the present invention relates to a wireless multi-modal tonoarteriogram monitoring apparatus and system based on nail sensing.

### BACKGROUND OF THE INVENTION

As people are more and more concerned about their own health condition, the application of wearable devices is becoming more and more popular among users, that is, users can monitor various physiological parameters of their body in real time through wearable devices, so that they can have an intuitive understanding of their body condition and make corresponding measures in time. However, most of the current wearable devices are worn on the wrists and fingers of users, which affects their normal life and makes it difficult to ensure long time monitoring.

### OBJECTS OF THE INVENTION

An object of the present invention is to provide a wireless multi-modal tonoarteriogram monitoring apparatus and system based on nail sensing.

The above object is met by the combination of features of the main claims; the sub-claims disclose further advantageous embodiments of the invention.

One skilled in the art will derive from the following description other objects of the invention. Therefore, the foregoing statements of object are not exhaustive and serve merely to illustrate some of the many objects of the present invention.

### SUMMARY OF THE INVENTION

Embodiments of the present invention provide a wireless multi-modal tonoarteriogram (TAG) monitoring apparatus and system based on nail sensing, which can solve the problem of how to perform long-term monitoring by wearable devices. The described technical solution is as follows. According to an aspect of an embodiment of the present invention, there is provided a nail sensing based, wireless multi-modal tonoarteriogram monitoring apparatus, the nail sensing based, wireless multi-modal tonoarteriogram monitoring apparatus, comprising a nail patch, a plurality of sensors and a processing module; at least one first sensor of the plurality of sensors is arranged on the nail patch, with the plurality of sensors being used for obtaining a biological signal at a fingernail; wherein the processing module is used for obtaining a target physiological parameter according to the biological signal at fingernail, the target physiological parameter comprising a target central tonoarteriogram signal.

As an optional embodiment of a nail sensing based, wireless multi-modal tonoarteriogram monitoring apparatus, further comprising a chip, wherein the processing module is optionally integrated at the chip.

As an optional embodiment of a nail sensing based, wireless multi-modal tonoarteriogram monitoring apparatus, wherein the processing module is used for: processing, in multi-modes, multi-wavelength photoplethysmogram (MWPPG) signals and an accelerometer signal to obtain an initial central tonoarteriogram signal corresponding to each mode; performing a channel estimation against the MWPPG signals according to a plurality of the initial central tonoarteriogram signals to obtain a channel estimation result; obtaining the target central tonoarteriogram signal according to the channel estimation result.

As an optional embodiment of a nail sensing based, wireless multi-modal tonoarteriogram monitoring apparatus, wherein the processing module processes, in multi-modes, the MWPPG signals and the accelerometer signal to obtain the initial central tonoarteriogram signal corresponding to each mode, comprising: inputting the MWPPG signals and the accelerometer signal into a pre-trained blood pressure prediction model to obtain the initial central tonoarteriogram signal outputted by the blood pressure prediction model, the blood pressure prediction model is trained by taking the MWPPG signals and the accelerometer signal as samples and referencing a central tonoarteriogram signal as a label; inputting the MWPPG signals and the accelerometer signal into a physiological mathematical model to obtain the initial central tonoarteriogram signal outputted by the physiological mathematical model.

As an optional embodiment of a nail sensing based, wireless multi-modal tonoarteriogram monitoring apparatus, wherein the processing module obtains the central tonoarteriogram signal according to the channel estimation result, comprising: inputting the channel estimation result into an aortic pressure estimation model based on dictionary learning to obtain the central tonoarteriogram signal.

As an optional embodiment of a nail sensing based, wireless multi-modal tonoarteriogram monitoring apparatus, wherein the biological signal comprises at least one of the MWPPG signals, a pressure signal and the accelerometer signal; the plurality of sensors comprise at least one of a MWPPG sensor, a pressure sensor and an accelerometer sensor; wherein the MWPPG sensor is used for obtaining the MWPPG signals; the pressure sensor is used for obtaining the pressure signal; and the accelerometer sensor is used for obtaining the accelerometer signal.

As an optional embodiment of a nail sensing based, wireless multi-modal tonoarteriogram monitoring apparatus, wherein the target physiological parameter further comprises a heart rate, a heart rate variability, a blood oxygen saturation level, a blood glucose level, a lactic acid value and a sleep parameter.

As an optional embodiment of a nail sensing based, wireless multi-modal tonoarteriogram monitoring apparatus, wherein the nail patch comprises a substrate layer and a protective layer layered in sequence; the substrate layer is adhered onto a nail and the substrate layer is made of a transparent material; the first sensor is arranged on the substrate layer, the processing module is optionally arranged on the substrate layer; the protective layer is made of a waterproof material.

As an optional embodiment of a nail sensing based, wireless multi-modal tonoarteriogram monitoring apparatus, wherein the biological signal comprises a pressure signal; the nail sensing based, wireless multi-modal tonoarteriogram monitoring apparatus further comprises a finger ring adjustable in expansion size; the plurality of sensors comprise a pressure sensor arranged on the finger ring, the pressure sensor being used for obtaining the pressure signal.

As an optional embodiment of a nail sensing based, wireless multi-modal tonoarteriogram monitoring apparatus, the said nail sensing based, wireless multi-modal tonoarteriogram monitoring apparatus further comprising a wireless communication module, the wireless communication module being arranged on the nail patch, wherein when the processing module is arranged on the nail patch, the wireless communication module is used for transmitting the biological signal to the processing module, and the wireless communication module is used for outputting the physiological parameter obtained by the processing module; wherein when the processing module is arranged to separate from the nail patch, the wireless communication module is used for transmitting the biological signal to the processing module.

In another aspect of an embodiment of the present invention, it provides a tonoarteriogram monitoring system, comprising the nail sensing based, wireless multi-modal tonoarteriogram monitoring apparatus and a wearable apparatus having a display screen; wherein, the wearable apparatus is used for displaying a pressure alert information at the display screen, the pressure alert information is used for indicating a strength of a target finger pressing on the display screen; the target finger being a finger wearing the nail sensing based, wireless multi-modal tonoarteriogram monitoring apparatus according to the first aspect. The technical solution provided by the embodiments of the present invention brings the beneficial effect that the present invention provides a nail sensing based, wireless multi-modal tonoarteriogram monitoring apparatus and system. Compared with the prior art by placing a finger cuff on a finger to obtain a biological signal, the invention uses the nail patch to adhere onto a nail which is light weight, good in appearance, and is convenient to wear by a user without affecting normal activities of the user which realizes a long-term monitoring of the user. At the same time, when compared with the placing of a finger cuff on a finger to obtain a biological signal, the use of a nail patch will not be affected by the pulling of skin to thereby obtain biological signal of higher quality.

### BRIEF DESCRIPTION OF THE DRAWINGS

The foregoing and further features of the present invention will be apparent from the following description of preferred embodiments which are provided by way of example only in connection with the accompanying figures, of which:

FIG. 1 shows a schematic diagram of a nail patch mounting position provided by an embodiment of the present invention;

FIG. 2 shows a schematic diagram of the structure of a nail sensing based, wireless multi-modal tonoarteriogram monitoring apparatus provided by embodiments of the present invention;

FIG. 3 shows a process flow schematic diagram when processed by a processing module provided by an embodiment of the present invention;

FIG. 4 shows a schematic diagram of a structure of a processing module provided by an embodiment of the present invention;

FIG. 5 shows a schematic diagram of a structure of a nail sensing based, wireless multi-modal tonoarteriogram monitoring apparatus provided by an embodiment of the present invention;

FIG. 6 shows a schematic diagram of a structure of a nail patch provided by an embodiment of the present invention;

FIG. 7 shows a schematic diagram of a structure of a nail patch provided by an embodiment of the present invention;

FIG. 8 shows a schematic diagram of a structure of a chip of a nail patch provided by an embodiment of the present invention;

FIG. 9 shows a schematic diagram of a structure of a chip of a nail patch provided by an embodiment of the present invention;

FIG. 10 shows a schematic diagram of a structure of a tonoarteriogram monitoring system provided by an embodiment of the present invention; and

FIG. 11 shows a schematic diagram of a structure of a tonoarteriogram monitoring system provided by an embodiment of the present invention.

### DESCRIPTION OF PREFERRED EMBODIMENTS

Embodiments of the present invention are described below in connection with the accompanying drawings in the present invention. It should be understood that the embodiments set forth below in connection with the accompanying drawings are exemplary descriptions for the purpose of explaining the technical solutions of the embodiments of the present invention and do not constitute a limitation of the technical solutions of the embodiments of the present invention.

It will be understood that the singular forms “one”, “a” and “the” as used herein may also include the plural forms, unless otherwise stated. It should be further understood that the terms “include” and “comprise” as used in the embodiments of this invention mean that the corresponding features can be implemented as the features, information, data, steps, operations, components and/or assemblies presented, but do not exclude the implementation of other features, information, data, operations, components and/or assemblies that are supported in the art. It should be understood that when we refer to a component being “connected” or “coupled” to another component, the component may be directly connected or coupled to the other component, or it may refer to the component and the other component being connected through an intermediate component. In addition, the “connection” or “coupling” as used herein may include wireless connection or wireless coupling. The term “and/or” as used herein indicates at least one of the items defined by the term, for example, “A and/or B” may be implemented as “A” or as “B”, or “A and B”.

In order to make the purpose, technical solutions and advantages of the present invention clearer, the embodiments of the present invention are described in further detail below in connection with the accompanying drawings.

The technical solutions of the present invention and the technical effects resulting from the technical solutions of the present invention are described below through the description of several exemplary embodiments. It should be noted that the following embodiments can be cross-referenced, borrowed or combined with each other, and the same terms, similar features and similar implementation steps, etc. in different embodiments will not be described repeatedly.

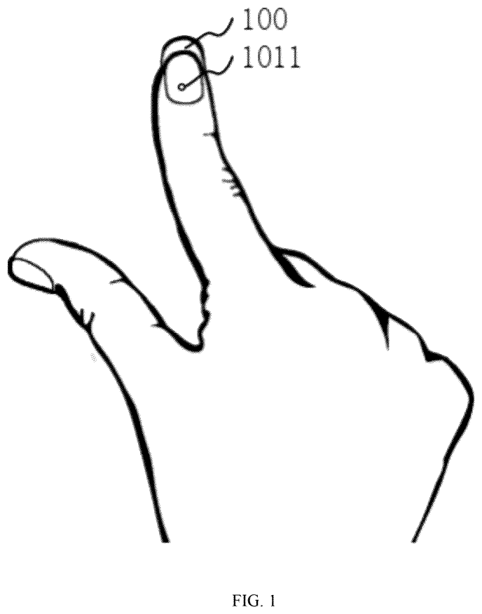

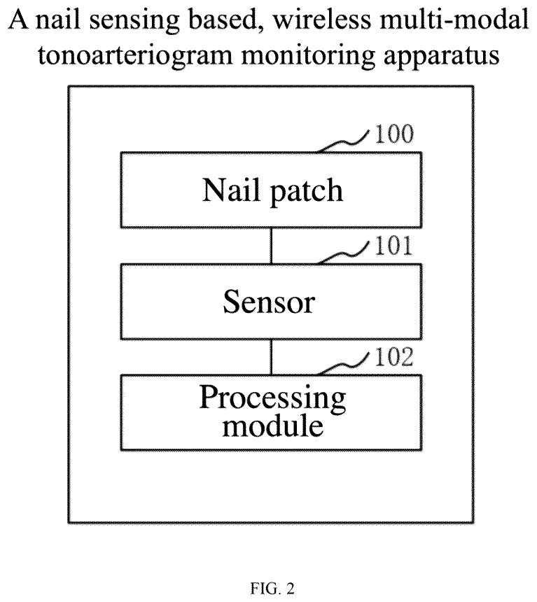

Embodiments of the present invention provide a nail sensing based, wireless multi-modal tonoarteriogram (TAG) monitoring apparatus, as shown in FIG. 1. FIG. 1 exemplarily illustrates a schematic diagram of a nail patch mounting position of an embodiment of the present invention. FIG. 2 exemplarily illustrates a schematic diagram of a structure of a nail sensing based, wireless multi-modal tonoarteriogram monitoring apparatus of an embodiment of the present invention and the nail sensing-based, wireless multi-modal tonoarteriogram monitoring apparatus comprises a nail patch 100, a plurality of sensors 101, and a processing module 102; at least one first sensor 1011 of the plurality of sensors 101 is arranged on the nail patch 100, with the plurality of sensors 101 being used for obtaining a biological signal at a fingernail; the processing module 102 is used for obtaining a target physiological parameter according to the biological signal at fingernail, the target physiological parameter comprising a target central tonoarteriogram signal. Wherein the nail patch 100 can be adhered to both the nail of the finger and the nail of the toe, and this implementation does not limit the specific location of the nail patch 100 placement.

Optionally, the biological information includes multi-wavelength photoplethysmogram (MWPPG) signals; the first sensor 1011 is multi-wavelength photoplethysmography (WMPPG) sensor, MWPPG sensor for obtaining MWPPG signals at the fingernail.

It should be explained that the multi-wavelength photoplethysmogram signals are detected by Photoplethysmography (PPG) to obtain the change curve of blood volume over time. The MWPPG sensor can emits multi-wavelength composite lights, and multi-wavelength composite lights are lights consisting of a variety of different wavelengths of monochromatic light. Because there are differences in the absorption and attenuation of each wavelength by blood, the MWPPG signals can be obtained when the sensor generates multi-wavelength composite lights (i.e., lights consisting of a variety of different wavelengths of monochromatic light). In addition, compared to the prior art where additional devices are placed on other body parts of the user to obtain MWPPG signals, direct generation of multi-wavelength composite lights by the WMPPG sensor can greatly reduce the number of components, miniaturize the nail sensing based, wireless multi-modal tonoarteriogram monitoring apparatus and improve the user's experience.

Of course, in other embodiments, the number of nail patch 100 has more than one and the number of first sensor 1011 also correspondingly can be set up more than one. Multiple first sensors 1011 respectively obtain different biological signals at fingernail, and the target physiological parameters are obtained by processing the biological signals from the plurality at fingernails through the processing module 102 to make the target physiological parameters more accurate.

Compared with the prior art by placing a finger cuff on a finger to obtain a biological signal, the invention uses the nail patch 100 to adhere onto a nail which is light weight, good in appearance, and is convenient to wear by a user without affecting normal activities of the user which realizes a long-term monitoring of the user. At the same time, when compared with the placing of a finger cuff on a finger to obtain a biological signal, the use of a nail patch 100 will not be affected by the pulling of skin to thereby obtain biological signal of higher quality.

Considering that the user can be monitored for a long time by the nail patch 100 placed on the nail, and the target central arterial tonoarteriogram signal is continuous blood pressure information. Compared with the intermittent blood pressure information obtained by devices such as cuffs in the prior art, which only includes systolic blood pressure (SBP) and diastolic blood pressure (DBP), the target central tonoarteriogram signal provides a more accurate assessment of the user's cardiovascular system and ensures the accuracy of the user's physical health assessment.

In addition, compared to the intermittent blood pressure information obtained by the finger instrument attached to the finger in the previous technology, which is blood pressure information at the fingernail, the target central tonoarteriogram signal is blood pressure information at the heart, and the useful information obtained by the target central tonoarteriogram signal is more accurate and abundant.

On the basis of the above embodiments, as an optional embodiment, the nail sensing based, wireless multi-modal tonoarteriogram monitoring apparatus further comprising a chip, wherein the processing module is optionally integrated at the chip.

That is, the response speed of obtaining the target central tonoarteriogram signal is improved by integrating the processing module on the chip, which also facilitates the integrated miniaturization of the nail sensing based, wireless multi-modal tonoarteriogram monitoring apparatus and further reduces the interference of the user's life.

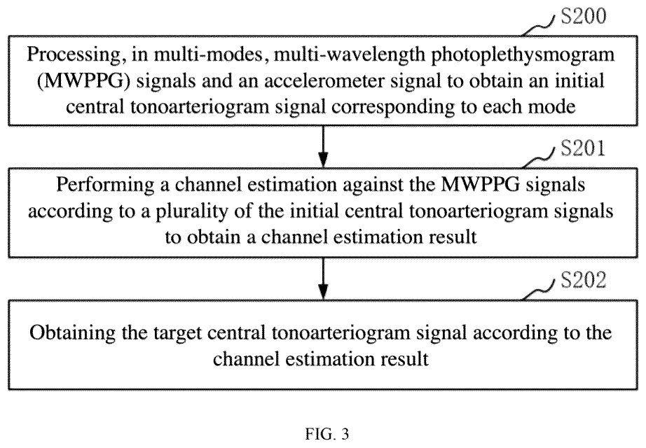

On the basis of the above embodiments, as an optional embodiment, as shown in FIG. 3, which exemplarily illustrates a process schematic diagram when processed by a processing module of an embodiment of the present invention, the processing module is specifically used to:
S200: processing, in multi-modes, multi-wavelength photoplethysmogram (MWPPG) signals and an accelerometer signal to obtain an initial central tonoarteriogram signal corresponding to each mode;
S201: performing a channel estimation against the MWPPG signals according to a plurality of the initial central tonoarteriogram signals to obtain a channel estimation result;
S202: obtaining the target central tonoarteriogram signal according to the channel estimation result.

It should be explained that, on the one hand, considering the existence of noise in the acquisition of MWPPG signals, the channel estimation of MWPPG signals is performed to make the obtained target central tonoarteriogram signal more accurate; on the other hand, considering the differences in the initial central tonoarteriogram signal obtained by different methods for MWPPG signals and accelerometer signal, and the advantages and disadvantages between multiple methods, the initial tonoarteriogram signal obtained by multiple initial tonoarteriogram signals can improve the accuracy of the channel estimation results, thus making the obtained target central tonoarteriogram signal more accurate.

In addition, considering that the noise generated by the MWPPG signals mainly come from the user's motion, the relationship between the user's current motion and blood pressure is determined by inputting accelerometer signal in the processing module to achieve the denoising of the MWPPG signals and improve the accuracy of the target central tonoarteriogram signal.

Based on each of the above embodiments, as an optional embodiment, the processing module processes, in multi-modes, the MWPPG signals and the accelerometer signal to obtain the initial central tonoarteriogram signal corresponding to each mode, comprising: inputting the MWPPG signals and the accelerometer signal into a pre-trained blood pressure prediction model to obtain the initial central tonoarteriogram signal outputted by the blood pressure prediction model, the blood pressure prediction model is trained by taking the MWPPG signals and the accelerometer signal as samples and referencing a central tonoarteriogram signal as a label; inputting the MWPPG signals and the accelerometer signal into a physiological mathematical model to obtain the initial central tonoarteriogram signal outputted by the physiological mathematical model. Wherein the reference central tonoarteriogram signal is obtained by cuff or invasive blood pressure measurement methods.

It can be understood that the blood pressure prediction model and the physiological mathematical model are two completely different methods for obtaining the initial central tonoarteriogram signal. The blood pressure prediction model is more specific in the initial central tonoarteriogram signal obtained because it involves training and samples, while the physiological mathematical model is the initial central tonoarteriogram signal obtained directly through mathematical equations, making the initial central tonoarteriogram signal obtained more general, and the accuracy of the channel estimation results can be improved by the combination of both.

On the basis of the above embodiments, as an optional embodiment, the processing module obtains the central tonoarteriogram signal according to the channel estimation result, comprising: inputting the channel estimation result into an aortic pressure estimation model based on dictionary learning to obtain the central tonoarteriogram signal.

It is understood that in other embodiments, the aortic pressure estimation model may also be based on other algorithms, which are not specifically limited in this embodiment.

That is, MWPPG signals are obtained from the fingernail vessel to obtain a target central tonoarteriogram signal, which in turn ensures more accurate monitoring of the user.

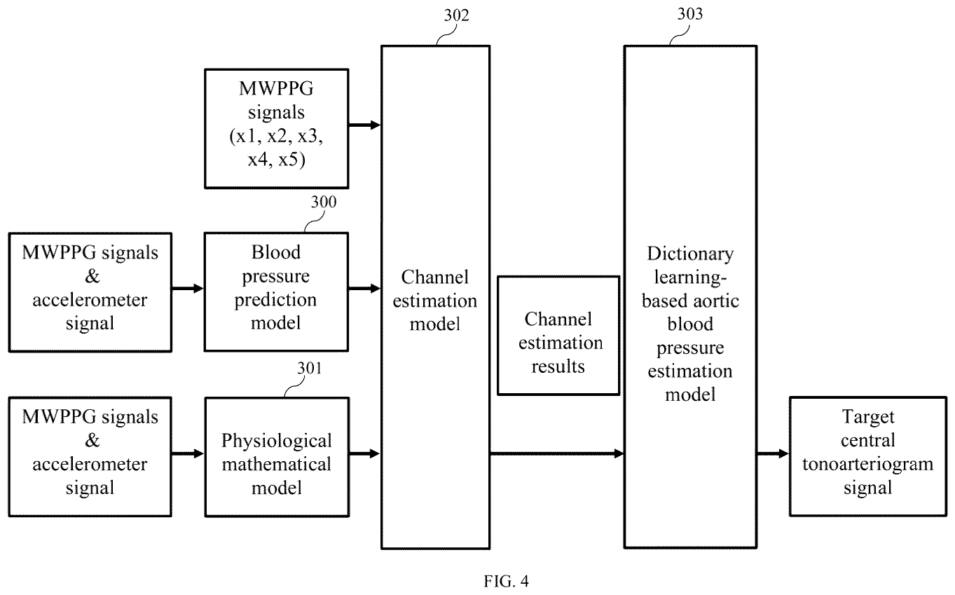

Based on the above embodiments, as an optional embodiment, a schematic diagram of the structure of a processing module is shown exemplarily in FIG. 4, wherein the processing module includes a blood pressure prediction model 300, a physiological mathematical model 301, a channel estimation model 302, and a dictionary learning-based aortic pressure estimation model 303, wherein the blood pressure prediction model 300 is used to obtain a corresponding initial central tonoarteriogram signal based on the MWPPG signals and the accelerometer signal, and the physiological mathematical model 301 is used to obtain a corresponding initial central tonoarteriogram signal based on the MWPPG signals and the accelerometer signal, the channel estimation model 302 is used to obtain the channel estimation results based on the MWPPG signals and the above two initial central tonoarteriogram signals, and the dictionary learning-based aortic blood pressure estimation model 303 is used to estimate the central arterial blood pressure based on the calibrated MWPPG signals and the accelerometer signal. The dictionary learning-based aortic blood pressure estimation model 303 is used to obtain a target central tonoarteriogram signal based on the calibrated MWPPG signals.

It is understood that in this embodiment, the MWPPG signals are obtained based on five different wavelengths, which are x1, x2, x3, x4, x5. Of course, in other embodiments, the number and type of wavelengths involved in the MWPPG signals can be adapted, and this embodiment is not specifically limited.

Based on each of the above embodiments, as an optional embodiment, the biological signal comprises at least one of the MWPPG signals, a pressure signal and the accelerometer signal; the plurality of sensors comprise at least one of a MWPPG sensor, a pressure sensor and an accelerometer sensor; wherein the MWPPG sensor is used for obtaining the MWPPG signals; the pressure sensor is used for obtaining the pressure signal; and the accelerometer sensor is used for obtaining the accelerometer signal. Wherein the MWPPG sensor and accelerometer sensor are set on the nail patch, and the pressure sensor can be set both on the nail patch and on other wearable devices, and this embodiment does not make specific limitations.

That is, the target physiological parameters are obtained by processing the MWPPG signals; the accelerometer signal is used to reduce noise and the hydrostatic pressure effect in the MWPPG signals to improve the accuracy of the target physiological parameters; considering that the target physiological parameters change with the user, the blood pressure prediction model and the physiological mathematical model may deviate from the user's own physiological condition, so the blood pressure prediction model and the physiological mathematical model are calibrated periodically by the pressure signal to ensure that the obtained target central tonoarteriogram signal can always be accurate.

It is important to explain that the pressure signal is a curve of transmural pressure over time obtained by applying a certain pressure to the vessel to flatten part of the vessel wall without causing vessel occlusion.

Based on each of the above embodiments, as an optional embodiment, the target physiological parameter further comprises a heart rate, a heart rate variability, a blood oxygen saturation level, a blood glucose level, a lactic acid value and a sleep parameter.

By obtaining a heart rate, a heart rate variability, a blood oxygen saturation level, a blood glucose level, a lactic acid value and a sleep parameter to achieve a comprehensive monitoring of the user's body, the accuracy of the user's health assessment is ensured.

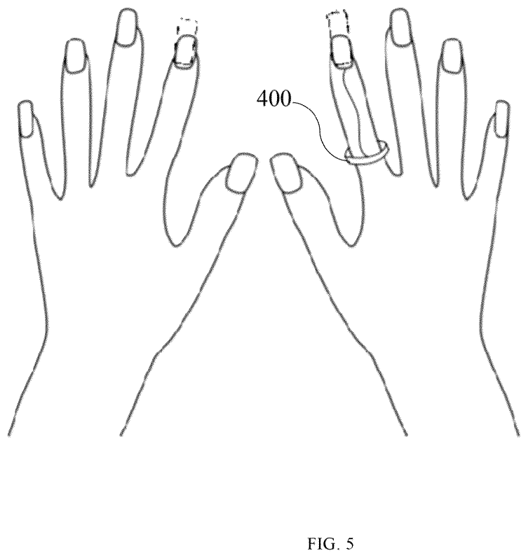

Based on each of the above embodiments, as an optional embodiment, a schematic diagram of the structure of a nail sensing based, wireless multi-modal tonoarteriogram monitoring apparatus is illustrated exemplarily in FIG. 5. The nail sensing based, wireless multi-modal tonoarteriogram monitoring apparatus further comprises a finger ring 400 adjustable in expansion size; the plurality of sensors comprise a pressure sensor arranged on the finger ring 400, the pressure sensor being used for obtaining the pressure signal.

It is understood that the blood pressure of the finger vessel with the finger ring is changed by the adjustment of the expansion size of the finger ring in order to be calibrated by the pressure signal, wherein the pressure signal combined with the hydrostatic pressure generated by the slow oscillation of the arm can obtain the oscillometric blood pressure value, which is used to calibrate the physiological mathematical model.

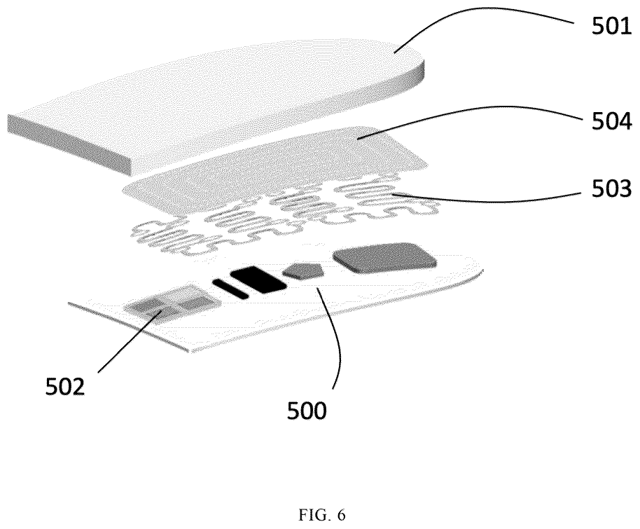

Based on each of the above embodiments, as an optional embodiment, a schematic diagram of the structure of a nail patch is exemplarily illustrated in conjunction with FIG. 6, wherein the nail patch comprises a substrate layer 500 and a protective layer 501 layered in sequence; the substrate layer 500 is adhered onto a nail and the substrate layer is made of a transparent material; the first sensor 502 is arranged on the substrate layer, the processing module 504 is optionally arranged on the substrate layer 500; the protective layer 501 is made of a waterproof material. Of course, in other embodiments, the processing module 504 may be arranged on other wearable devices. Wherein, the nail sensing based, wireless multi-modal tonoarteriogram monitoring apparatus further comprises a power supply module, the power supply module being provided on the substrate layer 500 for providing electrical power.

Optionally, when the nail sensing based, wireless multi-modal tonoarteriogram monitoring apparatus includes a finger ring adjustable in expansion size, power supply to the finger ring can also be achieved by the power supply module.

Further, the nail patch further includes a wiring layer 503 provided between the substrate layer 500 and the protective layer 501 for implementing an electrical connection between the first sensor 502, the power supply module, and the processing module 504.

Preferably, the substrate layer 500 is made of gallic acid modified organohydrogel combined with VHB tape, VHB tape has better adhesion compared to gallic acid modified organohydrogel, but VHB tape is less transparent compared to gallic acid modified organohydrogel, so the MWPPG sensor can be fixed at the gallic acid modified organohydrogel to ensure that the emitted light passes through the nail into the blood vessels as much as possible, to improve the quality of the obtained MWPPG signals. The accuracy of the MWPPG signals can be improved.

Preferably, the protective layer 501 is made of PVC or PE film, which has good waterproof sealing performance.

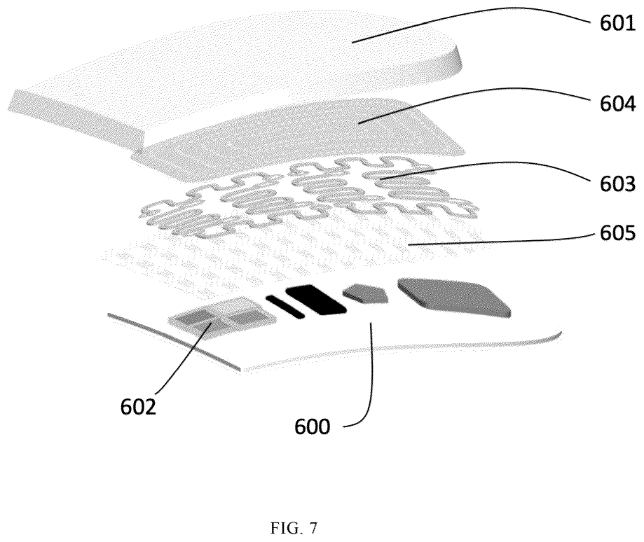

On the basis of the above embodiments, as an optional embodiment, in conjunction with FIG. 7, which exemplarily shows a schematic diagram of the structure of a nail patch, the nail patch includes sequentially laminated substrate layer 600 and protective layer 601, the substrate layer 600 is adhered to the nail, and the substrate layer 600 is made of transparent material; the first sensor 602 is arranged on the substrate layer 600, the processing module 604 is arranged on the substrate layer 600; the protective layer 601 is made of a waterproof material. Of course, in other embodiments, the processing module 604 may be arranged on other wearable devices. Wherein, the nail sensing based, wireless multi-modal tonoarteriogram monitoring apparatus further comprises a power supply module, the power supply module is arranged on the substrate layer 600 for providing electrical power.

Further, the nail patch further includes a wiring layer 503 provided between the substrate layer 600 and the protective layer 601 for achieving an electrical connection between the first sensor 602, the power supply module, and the processing module 604. Wherein, the plurality of sensors further includes a pressure sensor 605, the pressure sensor 605 is arranged between the wiring layer 603 and the base layer 600, and the pressure sensor 605 is a flexible pressure sensor.

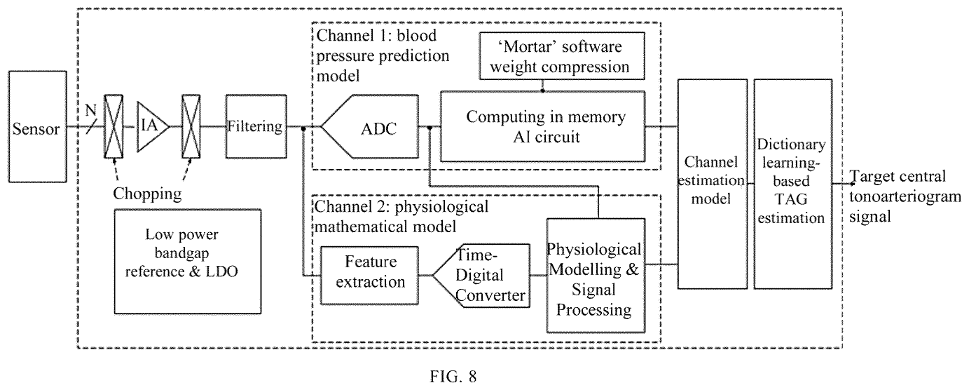

Based on each of the above embodiments, as an optional embodiment, when the processing module is integrated on the chip as shown in FIG. 8, the chip estimates the initial central tonoarteriogram signal by the blood pressure prediction model and the physiological mathematical model, respectively, and obtains the channel estimation results by the cross-relation channel estimation model to obtain better estimation performance. First, the sensor acquires differential signal from the N channels and after preprocessing to reduce the 1/f noise and common-mode offset and amplified with an IA. Filter is employed to extract the useful biological signal and further eliminate noise. Then, the signals go through 2 channels, wherein the input biological signal converted to digital signal via analog to digital converter (ADC), delivered to the AI Computing-in-Memory module that store all the initial central tonoarteriogram signal that trained by big dataset, with all the weight trained by software algorithm ‘Mortar’ to further decrease the computational overhead. The central tonoarteriogram signal is generated after MACs process of AI.

The biological signals in channel 2 are converted to digital signals by a time-to-digital converter and combined into a specific physiological mathematical model by a physiological module to output the initial central tonoarteriogram signal.

Finally, channel 1 and channel 2 are combined into a channel estimation model, and the target central tonoarteriogram signal is output by a dictionary learning-based aortic blood pressure estimation model.

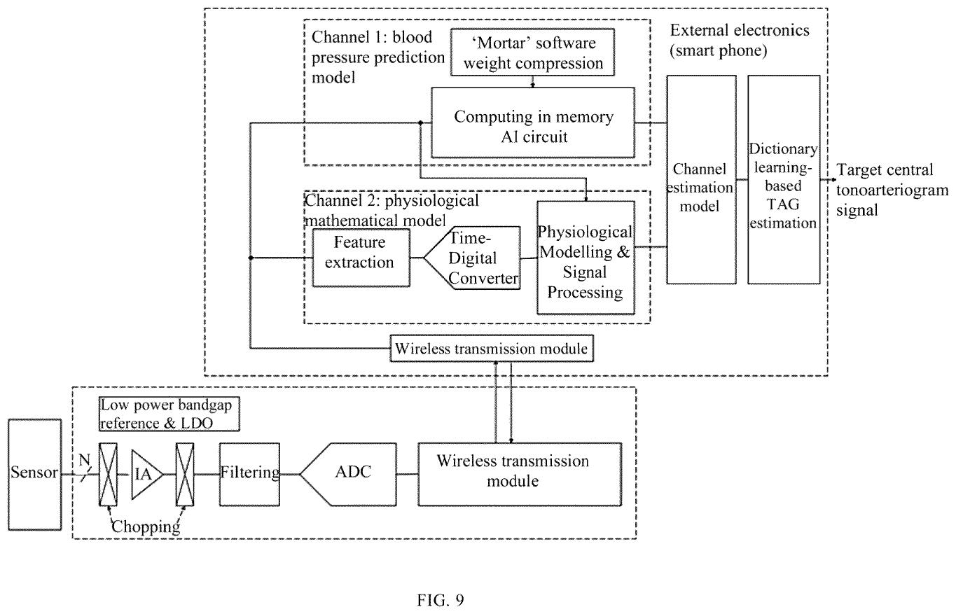

Based on each of the above embodiments, as an optional embodiment, when the wireless communication module is integrated on the chip and the processing module is separate and independent from the chip as shown in FIG. 9. The blood pressure prediction model and the physiological mathematical module are designed on an external display device, such as a cell phone, and the biological signal is transmitted to the cell phone via the wireless transmission module to obtain the target central tonoarteriogram signal and output.

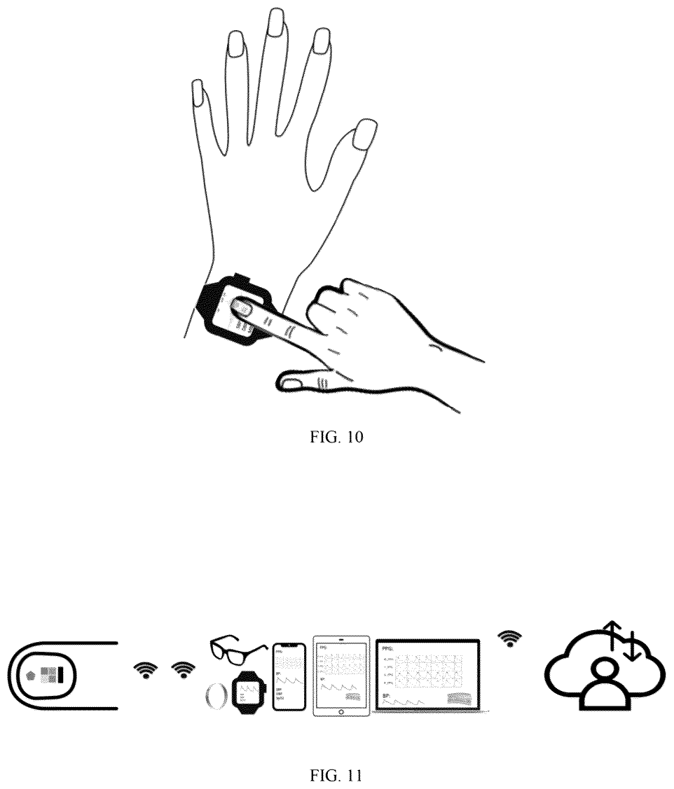

The present invention also provides a blood pressure map monitoring system, as shown in FIG. 10, the tonoarteriogram monitoring system includes the above-mentioned the nail sensing based, wireless multi-modal tonoarteriogram monitoring apparatus and a wearable apparatus having a display screen, wherein, the wearable apparatus is used for displaying a pressure alert information at the display screen, the pressure alert information is used for indicating a strength of a target finger pressing on the display screen, and the pressure is used to obtain the oscillometric blood pressure to calibrate the physiological mathematical model. The target finger is a finger wearing the above-mentioned the nail sensing based, wireless multi-modal tonoarteriogram monitoring apparatus, i.e., the pressure signal is obtained by pressing the wearable device, while the processing module measures the variable amplitude blood volume oscillation based on the pressure signal, and then calculates the systolic and diastolic blood pressure. After obtaining the oscillometric blood pressure values, they are applied to a physiological mathematical model for calibration.

Based on the above embodiments, as an optional embodiment as shown in FIG. 11, which exemplarily illustrates a schematic diagram of the structure of a tonoarteriogram monitoring system, the wireless communication module of the nail sensing based, wireless multi-modal tonoarteriogram monitoring apparatus includes a bluetooth for wirelessly connecting with other wearable devices and an external display device to output physiological parameters. The other wearable devices may be a cell phone, pad, computer, watch, glasses, etc., and the external display device may be a medical platform.

The terms “first”, “second”, “third”, “fourth”, “1”, “2”, etc. (if present) in the specification and claims of this invention and the accompanying drawings above are used to distinguish similar objects and need not be used to describe a particular order or sequence. It should be understood that the data so used is interchangeable where appropriate so that the embodiments of the present invention described herein can be implemented in an order other than that illustrated or described in the text.

It should be understood that although the individual operational steps are indicated by arrows in the flowcharts of embodiments of the present invention, the order in which these steps are performed is not limited to the order indicated by the arrows. Unless explicitly stated herein, in some implementation scenarios of embodiments of the present invention, the implementation steps in the respective flowcharts may be performed in other orders as desired. In addition, some or all of the steps in each flowchart may include multiple sub-steps or multiple stages based on actual implementation scenarios. Some or all of these sub-steps or phases may be executed at the same moment, and each of these sub-steps or phases may also be executed separately at different moments. In the scenario where the execution time is different, the order of execution of these sub-steps or stages can be flexibly configured according to the needs, and this invention is not limited in this regard.

It should be noted that for a person of ordinary skill in the art, other similar means of implementation based on the technical ideas of the present invention, without departing from the technical conception of the scheme of the present invention, also fall within the scope of protection of the embodiments of the present invention.

We claim:

1. A nail sensing based, wireless multi-modal tonoarteriogram (TAG) monitoring apparatus, comprising a nail patch, a plurality of sensors and a processing module; at least one first sensor of the plurality of sensors is arranged on the nail patch, with the plurality of sensors being used for obtaining a biological signal at fingernail;
wherein the processing module is used for obtaining a target physiological parameter according to the biological signal at fingernail, the target physiological parameter comprising a target central tonoarteriogram signal.

2. The nail sensing based, wireless multi-modal tonoarteriogram monitoring apparatus according to claim 1, further comprising a chip, wherein the processing module is optionally integrated at the chip.

3. The nail sensing based, wireless multi-modal tonoarteriogram monitoring apparatus according to claim 1, wherein the processing module is used for:
processing, in multi-modes, multi-wavelength photoplethysmogram (MWPPG) signals and an accelerometer signal to obtain an initial central tonoarteriogram signal corresponding to each mode;
performing a channel estimation against the MWPPG signals according to a plurality of the initial central tonoarteriogram signal to obtain a channel estimation result;
obtaining the target central tonoarteriogram signal according to the channel estimation result.

4. The nail sensing based, wireless multi-modal tonoarteriogram monitoring apparatus according to claim 3, the processing module processes, in multi-modes, the MWPPG signals and the accelerometer signal to obtain the initial central tonoarteriogram signal corresponding to each mode, comprising:
inputting the MWPPG signals and the accelerometer signal into a pre-trained blood pressure prediction model to obtain the initial central tonoarteriogram signal outputted by the blood pressure prediction model, the blood pressure prediction model is trained by taking the MWPPG signals and the accelerometer signal as samples and referencing a central tonoarteriogram signal as a label;
inputting the MWPPG signals and the accelerometer signal into a physiological mathematical model to obtain the initial central tonoarteriogram signal outputted by the physiological mathematical model.

5. The nail sensing based, wireless multi-modal tonoarteriogram monitoring apparatus according to claim 3, the processing module obtains the central tonoarteriogram signal according to the channel estimation result, comprising:
inputting the channel estimation result into an aortic pressure estimation model based on dictionary learning to obtain the central tonoarteriogram signal.

6. The nail sensing based, wireless multi-modal tonoarteriogram monitoring apparatus according to claim 1, wherein the biological signal comprises at least one of the MWPPG signals, a pressure signal and the accelerometer signal; the plurality of sensors comprise at least one of a MWPPG sensor, a pressure sensor and an accelerometer sensor; wherein the MWPPG sensor is used for obtaining the MWPPG signals; the pressure sensor is used for obtaining the pressure signal; and the accelerometer sensor is used for obtaining the accelerometer signal.

7. The nail sensing based, wireless multi-modal tonoarteriogram monitoring apparatus according to claim 1, wherein the target physiological parameter further comprises a heart rate, a heart rate variability, a blood oxygen saturation level, a blood glucose level, a lactic acid value and a sleep parameter.

8. The nail sensing based, wireless multi-modal tonoarteriogram monitoring apparatus according to claim 1, wherein the nail patch comprises a substrate layer and a protective layer layered in sequence; the substrate layer is adhered onto a nail and the substrate layer is made of a transparent material; the first sensor is arranged on the substrate layer, the processing module is optionally arranged on the substrate layer; the protective layer is made of a waterproof material.

9. The nail sensing based, wireless multi-modal tonoarteriogram monitoring apparatus according to claim 1, wherein the biological signal comprises a pressure signal; the nail sensing based, wireless multi-modal tonoarteriogram monitoring apparatus further comprises a finger ring adjustable in expansion size; the plurality of sensors comprise a pressure sensor arranged on the finger ring, the pressure sensor being used for obtaining the pressure signal.

10. The nail sensing based, wireless multi-modal tonoarteriogram monitoring apparatus according to claim 1, further comprising a wireless communication module, the wireless communication module being arranged on the nail patch,
wherein when the processing module is arranged on the nail patch, the wireless communication module is used for transmitting the biological signal to the processing module, and the wireless communication module is used for outputting the physiological parameter obtained by the processing module;
wherein when the processing module is arranged to separate from the nail patch, the wireless communication module is used for transmitting the biological signal to the processing module.

11. A tonoarteriogram monitoring system, comprising the nail sensing based, wireless multi-modal tonoarteriogram monitoring apparatus according to claim 1 and a wearable apparatus having a display screen;
wherein, the wearable apparatus is used for displaying a pressure alert information at the display screen, the pressure alert information is used for indicating a strength of a target finger pressing on the display screen;
the target finger being a finger wearing the nail sensing based, wireless multi-modal tonoarteriogram monitoring apparatus according to claim 1.
